(19) United States
(12) Patent Application Publication
(10) Pub. No.: US 2007/0016243 A1
(43) Pub. Date: Jan. 18, 2007

(54) Non-occlusive, retrievable dilation system

(76) Inventors: Venkatesh Ramaiah, Scottsdale, AZ (US); Robert Myron McNutt, Mesa, AZ (US)

Correspondence Address: SQUIRE SANDERS & DEMPSEY LLP, TWO RENAISSANCE SQUARE, 40 NORTH CENTRAL AVENUE, SUITE 2700, PHOENIX, AZ, 85004-4498, US

(21) Appl. No.: 11/478,340

(22) Filed: Jun. 28, 2006

Related U.S. Application Data
(60) Provisional application No. 60/595,378, filed on Jun. 28, 2005.

(51) Int. Cl. A61M 29/00

(52) U.S. Cl. 606/198

(57) ABSTRACT

A device for dilating either a vessel within a body or a structure positioned within the vessel is designed so that it does not occlude or substantially hinder the flow of blood through the vessel, thereby decreasing the windsock effect in blood vessels by maintaining fluid flow through the device during dilation. The device includes a plurality of wires that can be expanded from a first position wherein the device can be moved into or retrieved from the vessel, to a second position wherein it dilates the vessel or structure. When dilated, blood or other bodily fluid passes through the openings between the wires rather than being blocked.

### CROSS REFERENCE TO RELATED APPLICATIONS

This application claims the benefit of U.S. Provisional Application No. 60/595,378, filed Jun. 28, 2005, the contents of which are incorporated by reference herein.

### DESCRIPTION OF THE INVENTION

1. Field of the Invention

The present invention relates generally to medical devices, and more particularly to a medical device for the dilation of blood vessels and/or the dilation of structures positioned within blood vessels.

2. Background of the Invention

Conventional systems for dilating blood vessels and/or structures (e.g., a stent graph) positioned in a blood vessel utilize balloon-like structures. Such structures are made from essentially impermeable materials. When such a device is expanded to perform the dilation, blood flow is occluded through the blood vessel in which the balloon-like dilator is being used. Such an occlusion of blood flow may substantially or entirely harm the patient, since portions of the body will not receive blood during the procedure. Thus, the length of time balloon-like dilators may be used to perform dilations is limited.

Another problem with balloon-like dilators arises when a dilation procedure is being performed in a portion of the circulatory system where there is a branch in the blood vessels, such as where the arch vessels branch from the thoracic aorta. For example, improper placement of the balloon-like dilator in the aorta may cause an unanticipated occlusion in blood flow to a branch of the circulation system (in this example one of the arch vessels would be blocked). A further problem with impermeable balloon-like dilators is called the “windsock effect.” Because blood flow is substantially or entirely occluded when balloon-like dilators are in place, the blood pressure upstream of the dilator can be significant and may cause the balloon-like dilator, and any structure positioned in the blood vessel that was being dilated, to move out of the desired position, effectively pushed down stream (i.e., in the antegrade direction) by the blood. As such, accurate placement of such structures (e.g., stent grafts) can be difficult.

### SUMMARY OF THE INVENTION

The present invention provides a device for dilating either a vessel within a body (such as the human body) or a structure positioned within the vessel. The device is designed so that even when it is expanded it does not occlude or substantially hinder the flow of blood through the vessel. The device includes a plurality of wires that can be expanded from a first position in which the device can be moved into or retrieved from the vessel, to a second position in which the device is expanded and dilates the vessel and/or structure. When expanded (or dilated), blood or other bodily fluid passes through the openings between the wires rather than being blocked. The device may be used in any medical application in which dilation of a blood vessel or structure positioned within a blood vessel is desired (e.g., thoracic and abdominal aortic stent grafting).

A device according to the invention may have any suitable shape, structure or dimension, and may be expanded and contracted in any suitable manner.

According to one embodiment of the invention, the dilatation device is constructed as a spiraled mesh that can be expanded to dilate a vessel and/or structure within a vessel (e.g., to dilate an endograft and appose it to the aortic wall). The device can then be contracted to essentially its original size for removal from the vessel. In one embodiment, the expansion and unspiraling of the dilation device is accomplished using a twisting motion.

According to another embodiment of the invention, the dilation device is constructed as a non-spiraled, group of wires. The expansion and contraction of this dilation device is accomplished by applying linear pressure to the device, such as through a push/pull motion.

It is to be understood that the descriptions of this invention herein are exemplary and explanatory only and are not restrictive of the invention as claimed.

### BRIEF DESCRIPTION OF THE DRAWINGS

FIG. 1 shows examples of dilation devices according to various aspects of the invention.

FIGS. 2A-C shows a spiraled dilation device according to one embodiment of the invention.

FIGS. 3A-D shows additional view of a spiraled dilation device according to one embodiment of the invention.

FIGS. 4A-C shows a non-spiraled, expansive dilation device according to one embodiment of the invention.

FIGS. 5A-B shows another non-spiraled, expansive dilation device according to one embodiment of the invention.

FIGS. 6A-B shows a delivery and deployment system for a non-spiraled, expansive dilation device according to one embodiment of the invention.

FIG. 7 shows a control mechanism for a dilation device according to one embodiment of the invention.

### DESCRIPTION OF THE EMBODIMENTS

Reference will now be made in detail to the present exemplary embodiments of the invention, examples of which are illustrated in the accompanying drawings.

The present invention is a non-occlusive, retrievable dilation device for dilating blood vessels and/or structures positioned in blood vessels. The dilation device is designed so that it does not occlude or substantially hinder the flow of blood through the vessel (and, as used herein, the phrase “does not block,” when referring to blood flow, means that a device according to the invention does not occlude or substantially hinder the blood flow). Among the structures that may require dilation when placed in blood vessels are endografts, stents, stent grafts, and the like. The dilation device may be constructed in any suitable size to accommodate a particular blood vessel, including veins and arteries (e.g., abdominal aorta, aortic arch, ascending aorta, descending aorta, iliac arteries, or renal arteries). For example, the device may be used in wall apposition of thoracic and abdominal endoluminal grafts, which means it expands to position at least a portion of the graft snugly against the artery wall. The dilation device may introduced into a blood vessel either biaxially or triaxially (i.e., with a sheath or without) over a guide wire. Optionally, the dilation device includes one or more radio opaque markers that assist an operator in locating the device once in a vessel.

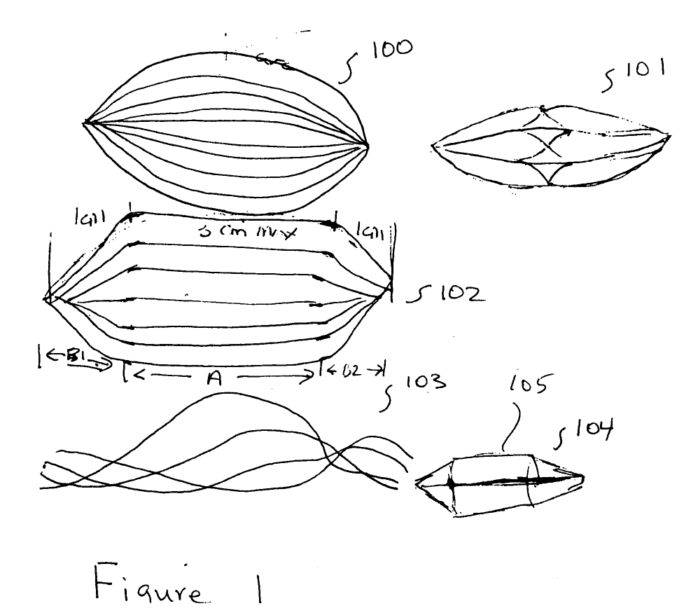

FIG. 1 shows examples of dilation devices according to various aspects of the invention. Each of the devices shown in FIG. 1 is constructed with a plurality of wires so that the flow of blood and/or other fluids are not occluded or seriously hindered when the device is in position in a vessel and expanded. Rather, fluids pass through the spaces between the wires and continue down the blood vessel (i.e., in the antegrade direction). The wires may be formed from any material suitable for use within blood vessels and able to be expanded from a first position that allows insertion into the blood vessel to a second position in which the device dilates the blood vessel and/or structure. Preferably, the dilation device is made from metal, such as stainless steel, nitinol, cobalt, chromium, or various other alloys.

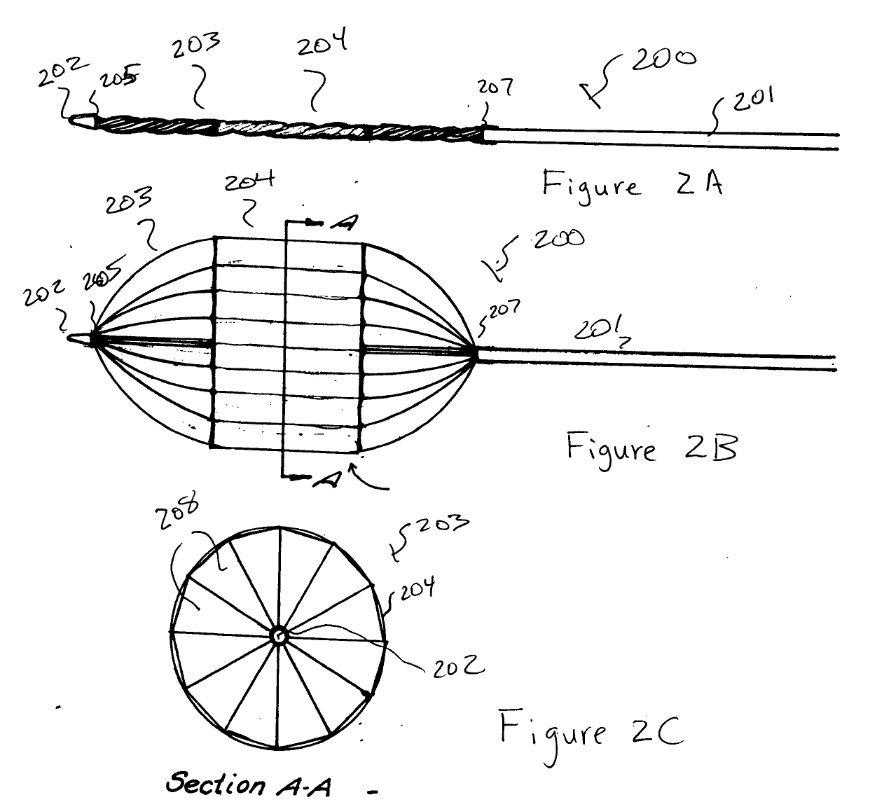

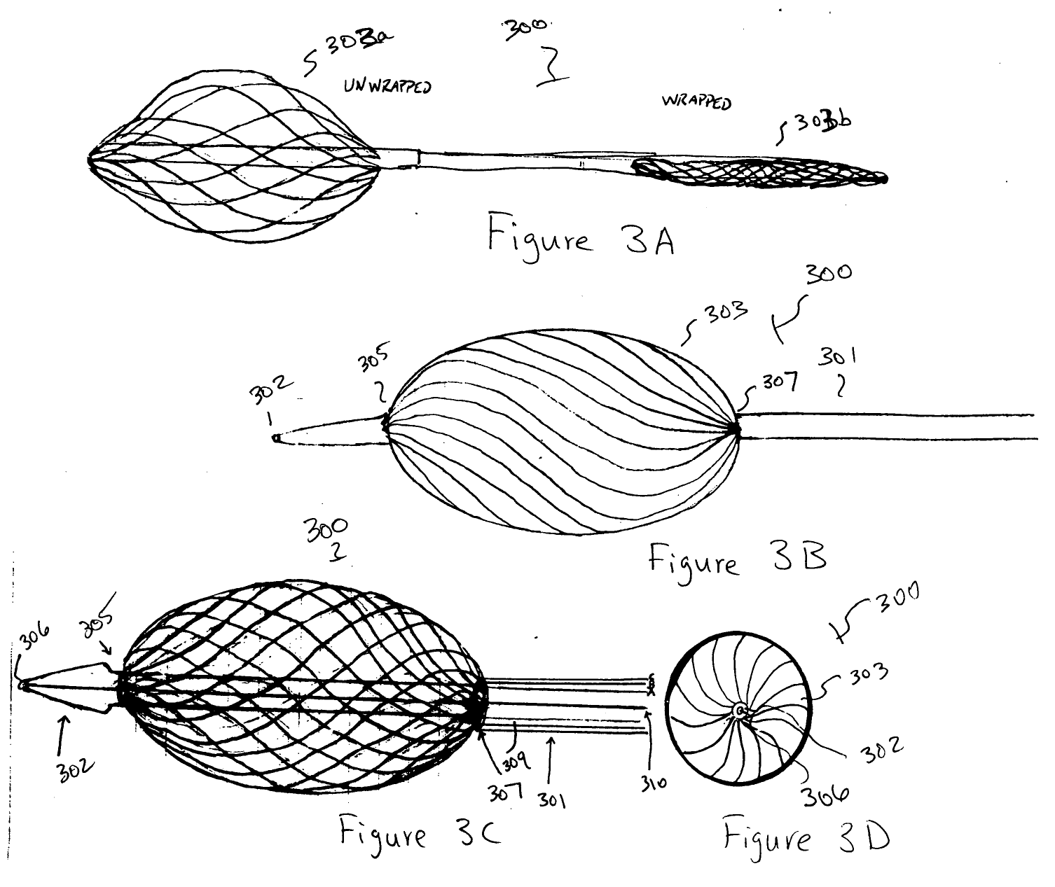

Returning to FIG. 1, device 100 shows a generally oval-shaped dilation device in an expanded position. The wires in device 100 have a substantially constant arc. Device 101 shows a generally oval-shaped dilation device with additional interconnected wires that form a cage-like structure. This cage-like structure provides for additional rigidity of the dilation device. Device 102 shows a dilation device with a substantially-linear section A of wires in the middle of the device, while the wires in end sections B1 and B2 are bent at angle so that they converge at approximately the same point at the ends. In this way, the dilation device may exert more even pressure against a blood vessel and/or device within the blood vessel along section A. In this example, the substantially-linear section is approximately 3 cm in length, while each of the end sections is 1 cm in length. However, the dilation device may be of any suitable size or shape and be constructed in any manner. Device 103 shows an exaggerated view of wires in dilation device 100 when in a spiraled position. In this position, the diameter of dilation device 100 is reduced, allowing for insertion into a blood vessel. Unspiraling the wires causes the device to expand, as shown in devices 100, 101, and 102. An embodiment for a spiraled dilation device will be discussed further with regard to FIGS. 2A-C and FIGS. 3A-D.

Device 104 shows a dilation device with a lining 105. Lining 105 may be positioned on part of the exterior surface and/or interior surface of device 104, or of any device according to the invention. The use of a lining (1) provides a more even surface for exerting pressure during the dilation process, thus better opposing a structure to the interior wall of a vessel in which the structure is located, and/or (2) prevents the wires in the device from becoming entangled with exposed wires on a stent or stent graft.

The lining is preferably made from a permeable material which would be important if the lining is positioned such that it could occlude blood flow (e.g., arch vessels like the carotid). However, impermeable materials may used when the lining is not positioned where it could seriously hinder blood flow. For example, in device 104, even if an impermeable material is used for the liner, blood will still flow through the gaps between the wires at each end of the device. Examples of preferable lining materials include polyurethane, PTFE (PolyTetraFluoroEthylene), nylon, or any material used in carotid embolic protection devices. However, any material suitable for use inside blood vessels may be used.

FIGS. 2A-C show a spiraled dilation device according to one embodiment of the invention. FIG. 2A shows a spiraled dilation device 200 in a first position for insertion into a blood vessel. Device 200 includes a catheter 201 with a distal tip 202. Catheter 201 has a central lumen running the length of the catheter to a wire port (not shown) in distal tip 202. Catheter 201 is inserted into a blood vessel over a guide wire going through the wire port in distal tip 202 and through the central lumen. In this context, catheter 201 may be any device having a central lumen and being capable of insertion into a blood vessel over a guide wire. Catheter 201 may be constructed in varying sizes to accommodate different blood vessels. Catheter 201 may be made of any material suitable for insertion into a blood vessel and capable of supporting a central lumen.

Dilation device 203 is affixed to catheter 201 near distal tip 202 at point 205 and at point 207. As shown in FIG. 2A, dilation device 203 is spiraled around the catheter is a first position. In this position, the catheter and dilation device are insertable into the blood vessel. Dilation device 203 may optionally include a lining 204 as discussed above with reference to FIG. 1.

FIG. 2B shows device 200 in an expanded position. Dilation device 203 is expanded by exerting a twisting motion on catheter 201. Because dilation device 203 is affixed at point 205 and at point 207, a twisting motion applied to catheter 201 will unspiral the device. The operation of the unspiraling mechanism will be discussed in more detail with reference to FIG. 3C. As can be seen in FIG. 2B, the use of optional lining 204 creates a substantially uniform surface for dilating blood vessels and structures.

FIGS. 2C shows a top view of section A-A when then dilation device 203 is in the expanded position. As can be seen in the top view, lining 204 provides for a more substantially uniform surface for dilating than would the wire mesh of dilation device 203 alone. Gaps 208 between the wires of dilation device 203 allow blood, and other fluids to flow through the device and down the blood vessel.

FIGS. 3A-D shows additional views of a spiraled dilation device according to one embodiment of the invention. FIG. 3A shows a spiral mesh structure rather than the straighter, cage-like structure of FIGS. 2A-C. In addition, the spiral mesh shown in FIG. 3A is denser than the structure shown in FIGS. 2A-C. The density of wires (i.e., the number and proximity of wires) used in the dilation devices may be varied for different applications. In general, the denser the wire mesh, the more uniform the expanded surface. Dilation device 303a is shown in the expanded or unwrapped position, while dilation device 303b is shown in the spiraled or wrapped position. FIG. 3B shows an expanded spiral mesh device in profile, including catheter 301, dilation device 303, affixation point 305, and distal tip 302.

FIG. 3C shows device 300 in more detail. Distal tip 302 is shown with a tapered front end. While not necessary, a tapered front end allows for easier insertion into a blood vessel if used biaxially or an additional sheath if used triaxially. At the end of distal tip 302 is a wire port 306 for insertion over a guide wire 310. The proximal end of distal tip 302 may have a reverse taper to affixation point 305. Affixation point 305 is the point at which the distal end of dilation device 303 connects to distal tip 302 of catheter 301. Affixation point 307 is the point at which the proximal end of dilation device 303 connects to secondary sheath 309. Secondary sheath 309 is positioned coaxially around catheter 301. Dilation device 303 is expanded by twisting secondary sheath 309. This is accomplished because the portion of dilation device 303 attached to secondary sheath 309 at affixation point 307 moves (i.e., twists), while the portion of dilation device 303 attached to distal tip 302 of catheter 301 at affixation point 305 remains stationary. As such, dilation device 303 unspirals (or unwraps) when secondary sheath 309 is twisted. FIG. 3D shows a top view of device 300.

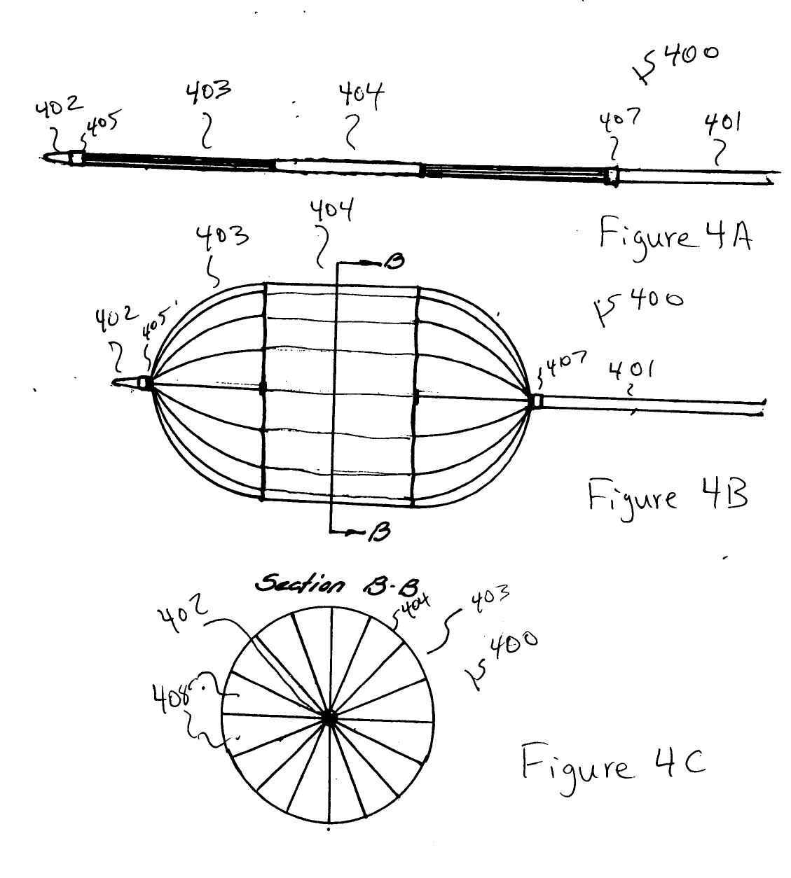

FIGS. 4A-C show a non-spiraled, expansive dilation device according to one embodiment of the invention. FIG. 4A shows a non-spiraled, expansive dilation device 400 in a first position for insertion into a blood vessel. Device 400 includes a catheter 401 with a distal tip 402. Catheter 401 has a central lumen running the length of the catheter to a wire port (not shown) in distal tip 402. Catheter 401 is inserted into a blood vessel over a guide wire going through the wire port in distal tip 402 and through the central lumen. In this context, catheter 401 may be any device having a central lumen and being capable of insertion into a blood vessel over a guide wire. Catheter 401 may be constructed in varying sizes to accommodate different blood vessels. Catheter 401 may be made of any material suitable for insertion into a blood vessel and capable of supporting a central lumen.

Dilation device 403 is affixed to catheter 401 near distal tip 402 at point 405 and at point 407. As shown in FIG. 4A, dilation device 403 is not spiraled around the catheter, but rather is affixed in a linear fashion in the first position. That is, each wire of dilation device 403 runs substantially linearly from affixation point 405 to affixation point 407. In this first position, the catheter and dilation device are insertable into the blood vessel. Dilation device 403 may optionally include a lining 404 as discussed above with reference to FIG. 1. As shown in FIGS. 4A-4C the lining is on the inside of dilation device 403.

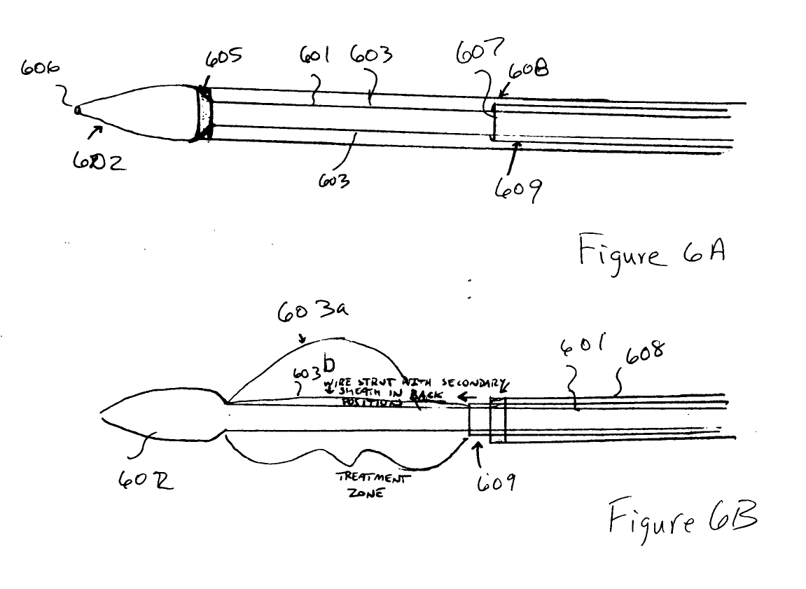

FIG. 4B shows device 400 in an expanded position. Dilation device 403 is expanded by exerting linear pressure on catheter 401 (e.g., a push-pull motion). Because dilation device 403 is affixed at points 405 and 407, a linear motion applied to catheter 401 will expand the device. The linear deployment mechanism will be discussed in more detail with reference to FIG. 6. As can be seen in FIG. 4B, the use of optional lining 404 creates a substantially uniform surface for dilating blood vessels and structures.

FIGS. 4C shows a top view of section A-A when then dilation device 403 is in the expanded position. As can be seen in the top view, lining 404 provides for a more substantially uniform surface for dilating. Gaps 408 between the wires of dilation device 403 allow blood, medicine, and other bodily fluids to flow through the device and down the blood vessel.

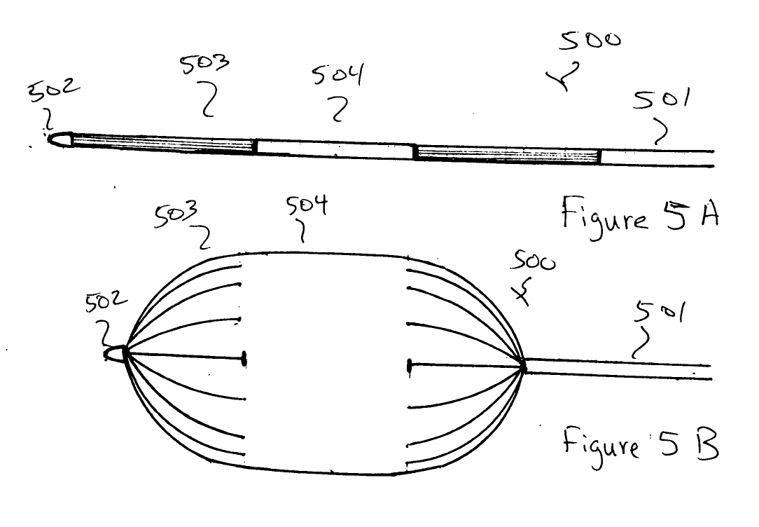

FIG. 5 shows another non-spiraled, expansive dilation device according to one embodiment of the invention. Device 500 is the same as device 400 except that liner 504 is placed on the outside of dilation device 503.

FIGS. 6A-B show a delivery and deployment system for a non-spiraled, expansive dilation device according to one embodiment of the invention. Catheter 601 includes a distal tip 602 with a wire port 602. Wire port 602 may be constructed to fit over any size guide wire (e.g., may be 0.038″ wire port). Again, distal tip 602 may be tapered at the tip for easier insertion into a blood vessel or addition sheath. Distal tip 602 may also be reversed tapered to affixation point 605. Affixation point 605 is where the distal end of dilation device 603 attaches to catheter 601. Secondary sheath 609 is positioned coaxially around catheter 601. The proximal end of dilation device 603 attaches to secondary sheath 609 at affixation point 607. An additional outer sheath 608 is positioned coaxially around catheter 601 and secondary sheath 609.

FIG. 6B shows the non-spiraled, expansive dilation device in two positions. In position 603a, dilation device 603 is expanded. The expansion is accomplished by pushing or screwing secondary sheath 609 forward. In this way, the proximal end of dilation device 603 is pushed forward while the distal end of dilation device 603 remains stationary because it is affixed to distal tip 602 of catheter 601. As such, the wires of dilation device 603 are pushed forward and expand to a predetermined maximum diameter. In position 603b, the wires of dilation device 603 remain at their smallest diameter. This position is achieved by pulling secondary sheath 609 back until distal tip 602 butts against outer sheath 608. Outer sheath 608 may include radiopaque markers to indicate when device has cleared the treatment zone.

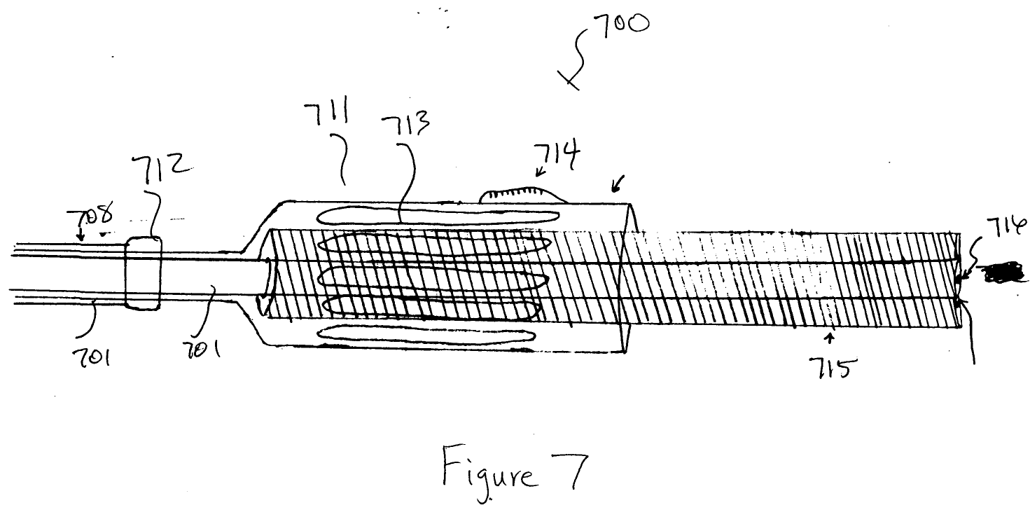

FIG. 7 shows a control mechanism for a dilation device according to one embodiment of the invention. Control mechanism is the hand-held portion of a dilation system and may be used with both spiraled and non-spiraled, expansive dilation devices. In the case of a non-spiraled, expansive dilation device, handle 711 is attached to outer sheath 708 through hemostatic value 712. For both spiraled and non-spiraled dilation devices, catheter 701 runs through handle 711 and has a wire port 716 at its proximal end. Handle 711 may include surface texturing 713 for easier grip. As shown in FIG. 7, handle 711 is a nut-type handle that is either fused to a secondary sheath and may be twisted (for a spiraled dilation device) or pushed/pulled (for a non-spiraled, expansive dilation device) to engage or disengage a dilation device. Handle 711 may also include a threaded, bolt-type fixation handle 715 fused to that is fused to catheter 701. This allows for execution of a twisting motion for spiraled dilation device. Handle 711 may also include a thumb-controlled quick release 714. Quick release 714 disengages handle 711 from the bolt-type fixation handle, allowing push/pull motions to be exerted on the handle and any attached sheaths and/or catheters (e.g., for engaging non-spiraled, expansive dilation devices.

Other embodiments of the invention will be apparent to those skilled in the art from consideration of the specification and embodiments disclosed herein. Thus, the specification and examples are exemplary only, with the true scope and spirit of the invention set forth in the following claims and legal equivalents thereof.

What is claimed is:

1. A device for dilating either a vessel within the body or a structure positioned within the vessel and for decreasing a windsock effect in blood vessels by maintaining fluid flow through the device during dilation, the device comprising a plurality of wires and movable from a first position wherein the device can be inserted into or removed from the vessel to a second position wherein the device dilates the vessel or structure within the vessel and wherein the device does not block blood flow when in the second position, whereby the windsock effect is decreased.

2. The device of claim 1 wherein the wire comprises metal.

3. The device of claim 1 wherein the wire comprises nitinol.

4. The device of claim 1 wherein the wire comprises stainless steel.

5. The device of claim 1 wherein the device further comprises two ends and a center portion, the center portion having an inner surface and at least part of the center portion including a lining on the inner surface.

6. The device of claim 1 that has an inner surface and that further includes a lining on part the inner surface.

7. The device of claim 1 wherein the device further comprises two ends and a center portion, the center portion having an outer surface and at least part of the center portion including a lining on the outer surface.

8. The device of claim 1 that has an outer surface and that further includes a lining on part the outer surface.

9. The device of claim 6 or 8 wherein the lining comprises PTFE.

10. The device of claim 6 or 8 wherein the lining is permeable.

11. The device of claim 6 or 8 wherein the lining is polyurethane.

12. The device of claim 6 or 8 wherein the lining is nylon.

13. The device of claim 1 wherein the vessel is an artery.

14. The device of claim 1 wherein the vessel is a vein.

15. The device of claim 13 wherein the vessel is the descending aorta.

16. The device of claim 13 wherein the vessel is the ascending aorta.

17. The device of claim 13 wherein the vessel is the abdominal aorta.

18. The device of claim 13 wherein the vessel is the aortic arch.

19. The device of claim 13 wherein the vessel is one of the iliac arteries.

20. The device of claim 13 wherein the vessel is a renal artery.

21. The device of claim 1 that is expanded from the first position to the second position by twisting it.

22. The device of claim 1 that is expanded from the first position to the second position by applying pressure to it.

23. The device of claim 1 wherein each of the wires forms a spiral when collapsed.

24. The device of claim 1 wherein the structure is a stent graft.

25. The device of claim 1 wherein the structure is a stent.

26. A catheter that includes an outer sheath, an inner sheath substantially coaxial with the outer sheath, and a device as described in claim 1, wherein the device is connected to the inner sheath.
